(19) United States
(12) Patent Application Publication
(10) Pub. No.: US 2003/0152984 A1
(43) Pub. Date: Aug. 14, 2003

(54) Method for the manufacture of DNA

(76) Inventors: Huseyin Aygun, Frankfurt am Main (DE); Markus Kircher, Frankfurt am Main (DE); Susann Rosmus, Frankfurt am Main (DE); Sylvia Wojczewski, Bad Soden (DE)

Correspondence Address: SHANKS & HERBERT, 1033 N. FAIRFAX STREET, SUITE 306, ALEXANDRIA, VA, 22314, US

(21) Appl. No.: 10/340,860

(22) Filed: Jan. 13, 2003

(30) Foreign Application Priority Data
Jan. 11, 2002 (EP) 02.000720.9

(51) Int. Cl. C12Q 1/68; C12P 19/34

(52) U.S. Cl. 435/6; 435/91.2

(57) ABSTRACT

The present invention relates to a method for manufacturing DNA comprising the steps of preparing n single stranded base-DNA-oligonucleotides which form immediately consecutive parts of the nucleotide sequence of the DNA being manufactured, in which the second to the n-th base-DNA-oligonucleotide is phosphorylated at the 5′ end and n is at least 2; preparing at least (n−1) single stranded joint-DNA-oligonucleotides capable of functioning as ligation templates for the base-DNA-oligonucleotides; contacting the base-DNA-oligonucleotides with the joint-DNA-oligonucleotides; subjecting the resultant product DNA-hybrid to a ligation reaction; and finally subjecting the resultant reaction product to an exonuclease reaction, in which the DNA strand of the reaction product formed by ligated base-DNA-oligonucleotides includes at least two cap-structures. Further the invention relates to DNA obtained by the method and a kit for carrying out the method.

Priority under 35 U.S.C. § 119, is hereby claimed to the following priority document: European Patent Application No. 02.000720.9, filed on Jan. 11, 2002.

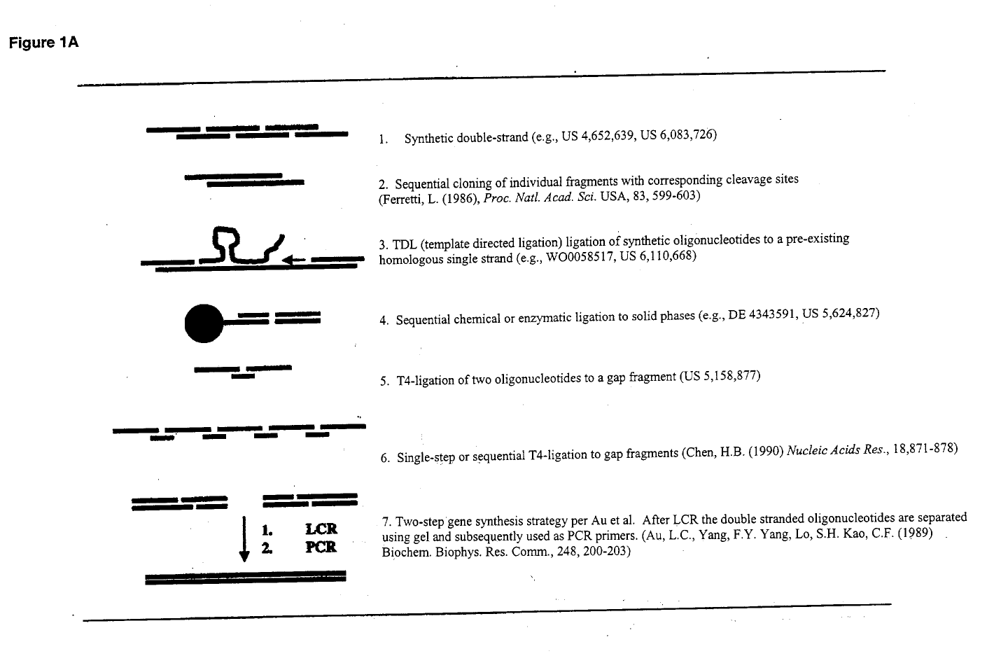

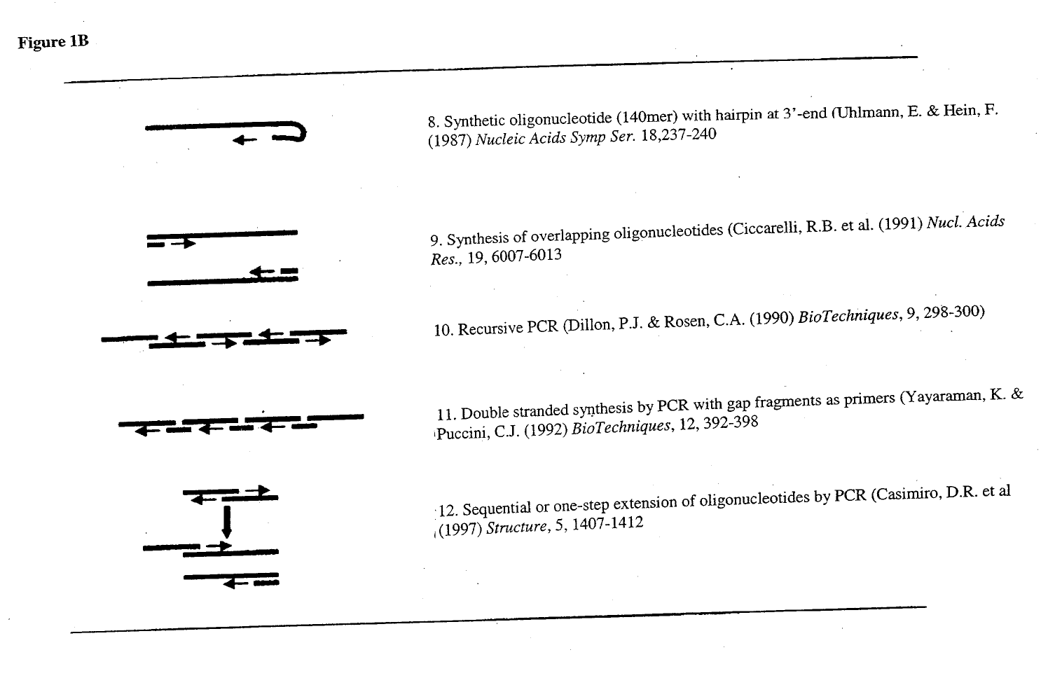

The present invention relates to a method in the field of nucleic acid synthesis. There exist at present a plethora of methods for synthesizing single or double stranded DNA (cf. FIGS. 1A and 1B).

The simplest type of single strand synthesis consists in chemically constructing single stranded DNA. Double stranded DNA may also be produced in this way through chemical synthesis of a strand (+) and complementary strand (−), followed by hybridization of both strands. This technique soon runs into difficulties, however. It is seldom possible to construct lengths of more than 150 base pairs employing standard DNA synthesis techniques. In addition, fragments and short strands occur that can only be effectively removed from the main product by very complex purification processes (gel electrophoresis).

Transforming single stranded DNA to a double strand may be accomplished by enzymatic means as well. In this case, external primers, for example, may be used to specifically amplify the intermediate region in a polymerase chain reaction (PCR). In another technique relatively long oligonucleotides are provided at their 3′ ends with hairpin configurations which position themselves as complements to one another and thus serve as “intramolecular” primers for an enzymatic extension (Uhlmann, 1987; see also FIG. 1B, No. 8).

Relatively long oligonucleotides also serve as the basis for another technique in which fully overlapping oligonucleotides, viz. double strands, are filled in a PCR using selected primers (Ciccarelli, 1991; see also FIG. 1B, No. 9).

However, the techniques described above are restricted in their use by the length of the oligonucleotides employed. Extension of gene synthesis to longer gene segments can be achieved by constructing so-called gene cassettes (U.S. Pat. No. 4,652,639, U.S. Pat. No. 6,083,726; see also FIG. 1A, No.1 and 2). Such gene cassettes consist of short, double stranded DNA fragments that can carry either select overhangs of from 3 to 7 base pairs (sticky end) or also smooth ends (blunt end) with 5′ phosphate groups. Overhangs are advantageous in that a given selection of these enables multiple fragments to be combined simultaneously during enzymatic ligation to form a gene. Such cassettes are also constructed by synthesizing single strands followed by hybridization of strand (+) and complementary strand (−). Prior to hybridization the 5′ phosphate groups are appended to the oligonucleotide by nucleotide kinases. Because ligation efficiency is low, it is frequently necessary when employing this strategy to clone intermediately individual gene fragments (Ferretti, 1986). Moreover, using degenerated oligonucleotides in conjunction with such a technique, e.g. for constructing DNA libraries, presents major difficulties.

Less complex are reactions that utilize PCR-based, sequential extension techniques (Ausubel, 1994; Jayaraman, 1991; Chang, 1993; Dillon, 1990; Jayaraman, 1992; Ye 1992). Also belonging to this category is, e.g. a method of synthesis described by Casimiro in 1997 (Casimiro, 1997; see also FIG. 1B, No. 12). In this method successive double strands of DNA are produced by amplifying single stranded oligonucleotides with complementary terminals in a PCR, which then serve as matrices (“templates”) for extending PCR-reactions. A serious drawback of this type of strategy is the accumulation of mutations resulting from the use of excessively high cycle counts in the PCR. In recursive gene synthesis (Dillon, 1990; Ausubel, 1994; Traub, 2001, see also FIG. 1B, No. 10), the genes being synthesized are constructed as overlapping strand (+) and complementary strand (−) oligonucleotides offset relative to one another. In a subsequent PCR the free 3′-terminal regions of these oligonucleotides may then be used as primers for synthesizing the complementary strand segment. With each extension new attachment sites become available for flanking sequences, thus allowing cycle by cycle synthesis of complete genes. This same strategy is also used in somewhat modified form in conjunction with another method. Instead of using long oligonucleotides in particular for extending a gene, here smaller gap fragments are used to combine individual gene fragments with one another. Such gap fragments function simultaneously as primers in a PCR for constructing the corresponding complementary strand (Yayaraman, 1992; see also FIG. 1A, No. 11). Just as in the method of Casimiro (Casimiro, 1997) a distinct disadvantage here is the accumulation of mutations resulting from the use of excessively high cycle counts in the PCR. Furthermore, the individual double stranded fragments inhibit efficient amplification of the full-length product since, because of their length, they hybridize with the template at significantly higher temperatures than the PCR external primers.

In still other gene synthesis strategies, the use of ligases is foremost (Sproat, 1985; Ferretti, 1986, Hostomsky, 1987; Wosnick, 1989; Climie, 1990; Oprian, 1991). In TDL-technology (“templated directed ligation”) oligonucleotides with 5′-phosphate groups are hybridized to a pre-existing single strand and subsequently linked enzymatically to oligonucleotide polymers (WO 0058517, U.S. Pat. No. 6,110,668; see also FIG. 1A, No. 3). A complementary strand of this type is obtained either as a result of prior exonuclease treatment of a corresponding wild type template or as a result of an asymmetrical PCR. This gene synthesis strategy is limited, however, to the production or reproduction of homologous genes. Using the T4-DNA ligase it is also possible to link pairs of oligonucleotides to one another with the aid of significantly shorter gap fragments (U.S. Pat. No. 5,158,877; see also FIG. 1A, No. 5). This type of ligation also presupposes a phosphate group at the 5′-end of the oligonucleotide located downstream (in the direction of the 3′-end). A variation of this method starts with a significantly greater number of single-stranded oligonucleotides that are eventually combined with one another by the T4-DNA ligase in one ligation step (Chen, 1990; FIG. 1A, No. 6). Ligation of this type is performed either in one step (all-in-one) or sequentially. A drawback of the single strand techniques is the lack of a complementary strand for suitable cloning in vectors. Chen, et al. was able to show, however, that direct cloning of single strands in previously opened vectors is entirely possible. Further, the use of the T4-DNA ligase restricts the ligation conditions to temperatures of around 37° C. As a result, ligation can be negatively effected by secondary structures that frequently arise in conjunction with long, single stranded oligonucleotides.

Other gene synthesis strategies combine the advantages of ligase- and/or polymerase-based partial steps (Au, 1998; Chalmers, 2001; see also FIG. 1A, No. 7). The proposed synthesis strategy of Au et al. (Au, 1998) starts with oligonucleotides complementary to one another (about 40 nucleotides), that are initially combined with the aid of thermally stable ligases (Pfu-DNA ligase) in a ligase-chain reaction LCR) to form double stranded partial fragments. These fragments are then isolated and recombined with one another through PCR.

In contrast to reactions that occur in solution are techniques based on gene construction on a solid phase (e.g. on beads) (U.S. Pat. No. 6,083,726, WO 9517413; see also FIG. 1A, No. 4). Linking to such a solid phase can be accomplished either through terminal modification of DNA with high alfin binding molecules (biotin, digoxigenin) or with functional groups (NH2, COOH, SH). The significant advantage of such synthesis strategies is that fragments not previously inserted during ligation can be removed in the subsequent wash steps. Based on a solid phase strategy of this type, sequential construction of larger genes can be accomplished either chemically (e.g. via 5′iodine- or 3′thiophosphate-modified oligonucleotides) or enzymatically, e.g. by repeated digestion with Alw261 (U.S. Pat. No. 6,083,726). When constructing with enzymes the T4-DNA ligase is used for blunt end or sticky end ligation of the double stranded gene fragment, or T4-DNA ligase is used to ligate single stranded oligonucleotides (WO 9517413).

An object of the present invention is to provide an advantageous method for manufacturing DNA.

It was unexpectedly found that the desired product or an intermediate product could be significantly enriched and/or selected by means of an exonuclease reaction following ligation to joint-like gap fragments.

Thus, the present invention relates to a method for manufacturing DNA that comprises a template-dependent ligation (“templage directed ligation”) to gap fragments and a subsequent exonucleasse reaction.

A first aspect of the invention is a method for manufacturing DNA that comprises the steps of:

a) preparing n single stranded base-DNA-oligonucleotides which form consecutive parts of the nucleotide sequence of the DNA being manufactured, in which

i) the second to the n-th base-DNA-oligonucleotide is phosphorylated at the 5′end and

ii) n is at least 2;

b) preparing at least (n−1) single stranded joint-DNA-oligonucleotides, applicable to which joint-DNA-oligonucleotide, the 3′terminal region of a joint-DNA-oligonucleotide is at least partially complementary to the 3′-terminal region of a base-DNA-oligonucleotide, and the 5′-terminal region of said joint-DNA-oligonucleotide is at least partially complementary to the 5′-terminal region of the immediately following base-DNA-oligonucleotide, such that when a joint-DNA-oligonucleotide is hybridized with 2 consecutive base-DNA-oligonucleotides a double stranded DNA-hybrid is formed in the region of the joint-DNA-oligonucleotide;

c) contacting the base-DNA-oligonucleotides with the joint-DNA-oligonucleotides;

d) subjecting the product DNA-hybrid from step c) to a ligation reaction;

e) subjecting the reaction product from step d) to an exonuclease reaction, in which the DNA strand of the reaction product of step d) formed by ligated base-DNA-oligonucleotides includes at least two cap-structures.

In a first step according to the method n single stranded base-DNA-oligonucleotides are prepared which form consecutive segments of the nucleotide sequence, and in which n is at least 2. The number n is preferably 3 to 100, more preferably 5 to 50 and most preferably 7 to 25.

The term “oligonucleotide” as used in the present application is not particularly limiting with regard to the length of the oligonucleotide. The base-DNA-oligonucleotides are normally from 45 to 1000 nucleotides in length, preferably from 50 to 500, more preferably from 75 to 300 and most preferably from 100 to 150 nucleotides. Base-DNA-oligonucleotides may be manufactured in a variety of ways. The standard manufacturing method, however, is to use the phosphoramidite-method for synthesizing oligonucleotides. The particulars of this method of synthesis and devices suitable for performing the method are known to those skilled in the art and may be found, for example, in Beaucage, S. L. & lyer, R. P. (1993) Tetrahedron, 49 (28), 6123-6194; Caruthers, M. H. et al. (1987) Methods in Enzymol., 154, 287-313; Beaucage, S. L. & Caruthers, M. H. (1981) Tetrahedron Lett. 22 (20), 1859-1862.

The “first” base-DNA-oligonucleotide is the most 5′ suitable base-DNA-oligonucleotide in the DNA being manufactured, relative to the strand, whose sequence matches the sequence of the base-DNA-oligonucleotide. The sequence of the “second” base-DNA-oligonucleotide attaches directly to the 3′end of the “first” base-DNA-oligonucleotide. The “n-th” or “last” base-DNA-oligonucleotide, is the most 3′ suitable base-DNA-oligonucleotide in the DNA being manufactured, relative to the strand, whose sequence matches the sequence of the base-DNA-oligonucleotide.

The base-DNA-oligonucleotides, with the expection of the first base-DNA-oligonucleotide, are phosphorylated at the 5′-end. This is required for subsequent ligation. Phosphorylation may be performed in a separate reaction following synthesis of the oligonucleotide. It is preferable, however, to perform phosphorylation in the DNA-synthesizer immediately at the end of oligonucleotide synthesis. The method is performed in ways known to those skilled in the art.

In a second step according to the method at least (n−1) single stranded joint-DNA-oligonucleotides are prepared. Generally, the joint-DNA-oligonucleotides are from 8 to 300 nucleotides in length, preferably from 10 to 100, more preferably from 16 to 70 nucleotides, and most preferably from 20 to 40 nucleotides. The joint-DNA-oligonucleotides as well are manufactured preferably using the phosphoramidite method.

Joint-DNA-oligonucleotides are oligonucleotides, which as a result of hybridization with 2 consecutive base-DNA-oligonucleotides can yield a DNA-hybrid having a double stranded and two single stranded regions. Thus, the joint-DNA-oligonucleotide fulfills the function of a ligation template, because it positions next to one another two consecutive base-DNA-oligonucleotides, so that given suitable conditions ligation may occur. The 3′-terminal region of a joint-DNA-oligonucleotide is thus at least partly complementary to the 3′-terminal region of a select base-DNA-oligonucleotide, and the 5′-terminal region of the same joint-DNA-oligonucleotide is at least partly complementary to the 5′-terminal region of the immediately following base-DNA-oligonucleotide, such that when hybridizing a joint-DNA-oligonucleotide with 2 consecutive base-DNA-oligonucleotides, a double-stranded DNA-hybrid is formed in the region of the joint-DNA-oligonucleotide.

The degree of complementarity need not be 100%, but it must be sufficient in order to ensure hybridization under suitable conditions. A match of at least 95% is preferred. In a preferred embodiment the degree of complementarity is 100%.

The length of the region of the joint-DNA-oligonucleotide hybridized with a selected base-DNA-oligonucleotide is dependent primarily upon the total length of the joint-DNA-oligonucleotide. Ordinarily, the 5′-terminal half of a joint-DNA-oligonucleotide may hybridize with one base-DNA-oligonucleotide, and the 3′-terminal half of the joint-DNA-oligonucleotide with another. However, deviations from such half divisions are entirely possible.

In a separate embodiment the joint-DNA-oligonucleotides are modified in such a way that they cannot be enzymatically extended at the 3′-end, e.g. using DNA-polymerases.

In a third step according to the method the base-DNA-oligonucleotides are contacted with the joint-DNA-oligonucleotides. This occurs under conditions that allow hybridizations to occur between the joint-DNA-oligonucleotide(s) and the base-DNA-oligonucleotides.

In still a further step according to the method the product DNA-hybrid from the previous step is subjected to a ligation reaction. This step may also be performed substantially in conjunction with the third step, that is, the various oligonucleotides are simply mixed together with the ligation reagents and incubated under conditions that allow ligation to occur.

For ligating, it is feasible to use various enzymes exhibiting ligase activity, for example, T4-DNA-ligase which exhibits the highest degree of activity in a temperature range of between 16° C. and 37° C. It has proved especially advantageous, however, to use a thermostable ligase. By this means it is possible to obtain solid ligation yields at elevated temperatures even for long base-DNA-oligonucleotides (>150 nucleotides in length). Preferred enzymes are Taq DNA-ligase and Pfu DNA-ligase.

It is essential to the method according to the present invention that the reaction product of the ligation reaction be subjected in a fifth step to an exonuclease reaction. “Exonuclease” as used in the present application is an enzyme that cleaves nucleotides sequentially from free ends of a linear nucleic acid substrate. By contrast, an “endonuclease” cleaves the nucleic acid substrate at internal sites in the nucleotide sequence.

Such a reaction may directly follow ligation, though it is conceivable to have intermediate steps occurring between ligation and exonuclease treatment. After ligation the reaction product may be isolated or enriched, for example, through precipitation of the DNA. However, it is also conceivable that the reaction mixture be subjected in essentially unaltered form to exonuclease treatment.

For exonuclease treatment enzymes exhibiting exonuclease activity are used. Potential enzymes are, for example, exonuclease VII, general exonucleases, preferably exonuclease VII, but also exonuclease I, exonuclease III and exonuclease V, as well as DNase and mixtures of the aforementioned hydrolases.

The DNA strand of the reaction product formed by ligated base-DNA-oligonucleotides contains at least two cap structures. A “cap structure” as used in the present application is a structure that lends resistance to an exonuclease at one end of a linear nucleic acid. In this way the desired DNA-sequence being synthesized is protected from nuclease degradation. A first cap-structure is located in the 5′-terminal region of a DNA-sequence being synthesized, while a second cap-structure is located in the 3′-terminal region of said DNA sequence being synthesized.

The cap structure may, but need not be, located at the immediate 5′- or 3′-end of the DNA-strand of the reaction product formed by ligated base-DNA-oligonucleotides. The present invention also encompasses the case in which one or two ends of said strand have nucleotides that are unprotected against exonuclease degradation. What is essential is that the desired DNA-sequence be protected by cap-structures. Thus, further encompassed by the present invention is the case in which nucleotides are introduced through base-DNA-oligonucleotides at the ends of the existing DNA-strand that need not be contained within the desired DNA-sequence. Nucleotides of this type need not be protected from nuclease degradation.

Various cap-structures are known to those skilled in the art. Examples of these are thioate bonds between individual nucleotides, 2′Omethyl-RNA, modified bases, DNA-sequences with loop structure(s) and/or RNA sequences with loop structure(s). Base modifications that protect against exonuclease degradation are C-5 propinyl or C-5 methyl-modified bases, 2-amion-2′-deoxy adenine, N-4-ethyl-2′-deoxy cytidine, 2′-deoxy inosine, 2′-deoxy uridine, as well as the unnatural bases nebularine, nitropyrrol and 5-nitroindole.

There are also other 3′ and 5′ modifications that protect against nuclease degradation, such as primary, secondary and tertiary amines which, like hydroxyl- and thiol-groups, append from terminal phosphate groups (3′ and 5′ phosphate) by way of aliphatic linkers or aliphatic linkers modifed by oxygen “O”, sulfer “S” or nitrogen “RR′R″N”, branched or straight ethylene glycole, the same as glycerin derivatives. End-position markers such as biotin, dinitrophenol, and digoxigenine may also be used, in addition to all commercial dyes directly obtainable in the form of phosphoramidites or indirectly as active esters.

Generally, a first cap-structure is introduced by the first base-DNA-nucleotide, a second cap-structure is introduced by the n-th-base-DNA-oligonucleotide. It would also be theoretically conceivable for base-DNA-oligonucleotides located further in to also include a cap-structure, though this is not preferred.

The reaction product of the exonuclease treatment is a singles tranded DNA with cap-structures at each end. In accordance with a separate embodiment of the present invention this single stranded DNA may be transformed by PCR into double stranded DNA and propagated. To this end it is preferable to use primers whose target sequence are located in the 5′-terminal region or in the 3′-terminal region of the desired DNA sequence. Normally, the target sequences are located in the region of the first or last base-DNA-oligonucleotide. With the aid of primers it is also possible to introduce restriction cleavage sites at the terminal regions of the double stranded DNA, the primers containing a recognition sequence for one or more restriction endonucleases. The double stranded DNA product manufactured in this way may then be digested by restriction enzymes and, for example, cloned in a plasmid or a vector, at which point the DNA may then be introduced into a cell. In this way the manufactured DNA may, for example, be propagated in bacteria. Techniques of this kind are known to those skilled in the art. The DNA may also be introduced into eukaryotic cells, e.g. mammalian cells in order to express the desired polypeptides.

In one embodiment of the present invention one or more base-DNA-oligonucleotides and/or joint-DNA-oligonucleotides contain randomized nucleotides. As a result, it is possible to manufacture DNA that exhibits variations at select positions. Such variations are already incorporated in a sequence during oligonucleotide synthesis. By using DNA-phosphoramidite mixtures which, instead of individual phosphoramidites, contain all bases (dA, dC, dG and dT) in select proportions (N-mixtures), partial or completely randomized oligonucleotides are obtained. Such oligonucleotides may be perfected to become complete genes by the method described herein, and they provide the desired protein or peptide libraries incorporated in the corresponding vectors. Such libraries form the basis for the search for selected, novel character patterns. Adding an N-mixture to the individual monomers (XN-mixtures) also provides the possibility of restricting the degree of randomization. This ensures that the breadth of variation within a protein or peptide library remains small in proportion to the starting gene. This strategy prevents existing positive mutations from being suppressed or lost through superimposition with other mutations in the protein or peptide library.

A further aspect of the present invention involves DNA obtained by the method described herein, in particular DNA that has been manufactured in accordance with this method. The invention further relates to a DNA-hybrid comprising a single strand DNA, one or more joint-DNA-oligonucleotides hybridized with it and at least two cap-structures.

Further the present invention relates to a kit suited to carrying out the method. The kit of the present invention contains a first base-DNA-oligonucleotide that includes a cap-structure, a second base-DNA-oligonucleotide that includes a cap-structure, an enzyme exhibiting ligase activity and an enzyme exhibiting exonuclease activity. Said kit may also include reagents for use in implementing the method, such as concentrated buffer solutions.

Still further, the kit may also contain means for performing a PCR, such means being, for example primers and a thermostable DNA-polymerase. The primers contain preferably one or more recognition sequences for one or more restriction endonucleases.

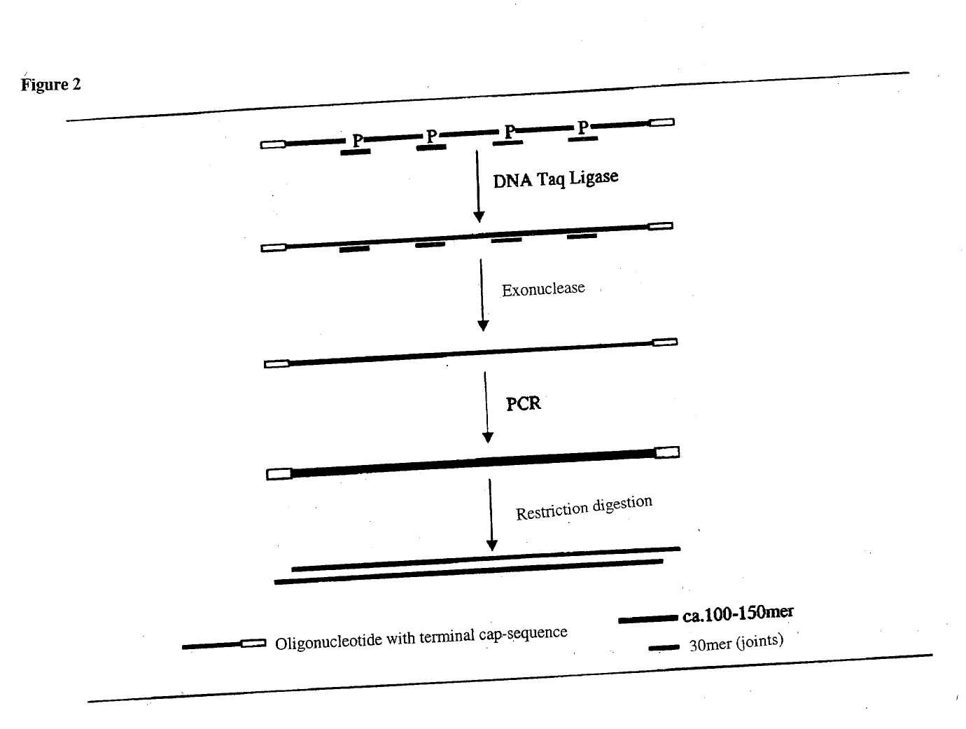

The present method for complete, chemo-enzymatic gene synthesis (cf. FIG. 2) is distinguished by numereous advantages over and against conventional methods:

It permits the complete synthetic construction of genes based on particularly long base-DNA-oligonucleotides (45-1000 base pairs). There is no complementary strand synthesis, which significantly reduces the time expenditure and costs involved in constructing genes or gene clusters. Unlike other gene synthesis strategies, it is possible to forego the time-consuming intermediate cloning of gene fragments. Moreover, constructing a single strand alone allows for the introduction of mutations at the level of synthetic DNA. Mutagenized or randomized segments may thus be generated at any site on the gene and perfected during complementary strand synthesis (PCR). This avoids difficulties encountered in hybridization of randomized sequences.

A unique feature of the method according to the present invention is the use of cap-structures, in particular of 5′ and 3′ overhangs, for in vitro selection of ligation products. Such cap-structures consist of 3′ or 5′ nuclease resistances that can not be shorted by polymerases or enzymes exhibiting nuclease activity (5′→3′ and 3′≦5′). The full-length product resulting from ligation is protected at both ends against nuclease degradation, but all shorter intermediate products or inserted oligonucleotides, including end-position nucleotides, are not. In this way the full-length product, which is protected at both ends following nuclease treatment, is selected or significantly enriched in the reaction preparation. Subsequent conventional PCR then produces the desired double stranded gene product.

The synthesis and purification protocols may be modified to obtain particularly long oligonucleotides of high quality and precision. Moreover, the use of a special phosphorylating reagent allows for the separation of terminally modified base-DNA-oligonucleotides only, thus making the oligonucleotide-specific ligation (OSL) very efficient.

Factors such as for example, codon usage, may be optimally adapted to the respective host as early as the oligonucleotide construction phase, which in part makes the expression of heterologous proteins possible in the first place.

The target gene or gene cluster is assembled enzymatically with the aid of short complementary oligonucleotides (joints), which function as ligation templates. This eliminates the need for a complete gene strand for select ligation of base-DNA-oligonucleotides. Undesirable effects of such joints in subsequent PCR may be checked through the use of 3′phosphate groups that are unextendable by enzymatic means.

For OSL it is feasible, in addition to conventional ligases, such as for example, the T4 DNA ligase (16° C. to 37° C.), to use thermostable ligases, such as for example, Taq or Pfu DNA ligase (37° C.-80° C.). Frequently, this enables one to determine optimal ligation conditions.

The total synthesis of target genes using the method presented herein provides new options in the assembly of gene libraries:

i) For example, specific amino acids or sequence segments may be fully randomized at the DNA-synthetic level and thus be introduced into the gene.

ii) Otherwise, with the aid of this technology one can also generate “restrictively-randomized” sequences, which for example, only permit hydrophobic amino acids, permitted in the gene (e.g. NTN).

FIG. 1A and 1B illustrate in schematic form the fundamentals of various methods for manufacturing DNA (see also above).

FIG. 2 illustrates in schematic form a selected embodiment of the method according to the present invention. Here, five base-DNA-oligonucleotides are prepared, of which the first and fifth each contain a cap-structure. The base-DNA-oligonucleotides two to five are pohsphorylated at the 5′-terminus. Serving as ligation templates are four joint-DNA-oligonucleotides shown beneath the gap sites of the base-DNA-oligonucleotides. The base-DNA-oligonucleotides are joined in ligation to form a single strand to which the joint-DNA-oligonucleotides are then hybridized. The latter are then degraded by the exonucleases, while the ligated single strand is protected by the cap-structures. The single strand is then transformed by PCR into double stranded DNA. Cleavage sites are introduced in PCR in order to allow for restriction digestion.

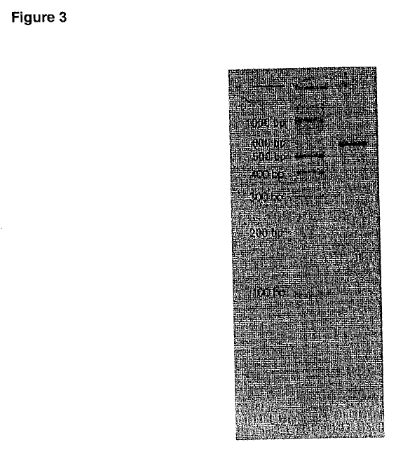

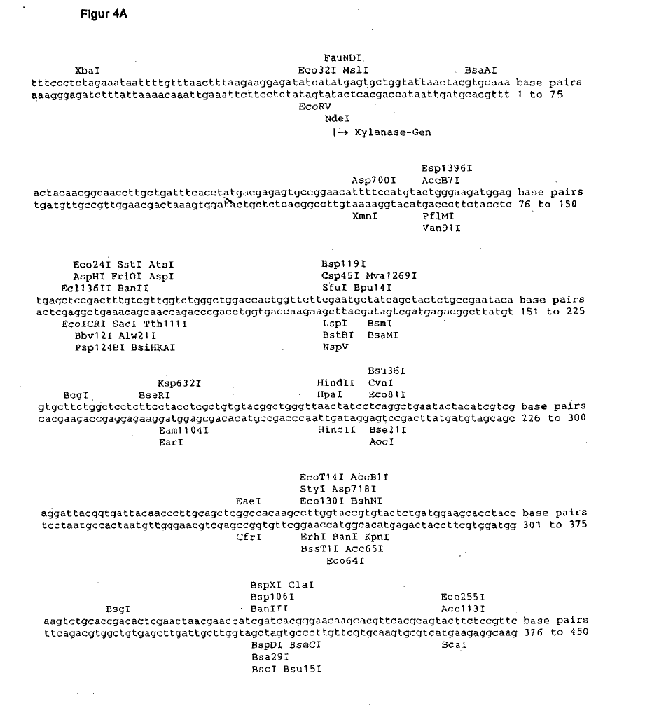

FIG. 3 shows the xylanase gene manufactured in Example 1 after ligation by Taq-ligase, exonuclease treatment and PCR amplification. To the left is a 100 pb marker (New England Biolabs) coating, to the right 10 μl of the amplification preparation on a 2% agarose gel in 1× TBE.

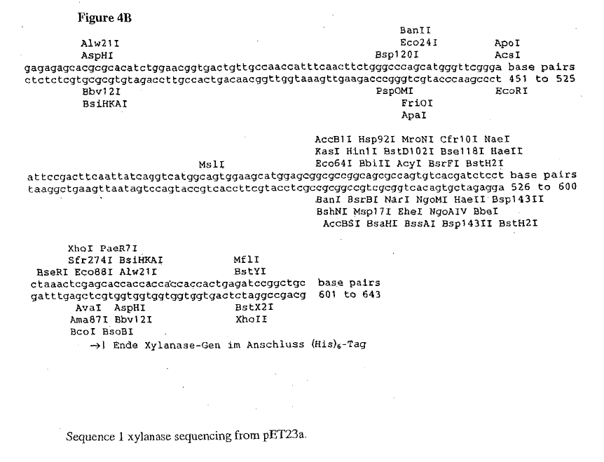

FIGS. 4A and 4B show the nucleotide sequence, identified through DNA-sequencing, of the DNA manufactured in Example 1 after cloning in the pET 23a vector. The identified sequence for the xylanase gene is identical to the desired sequence.

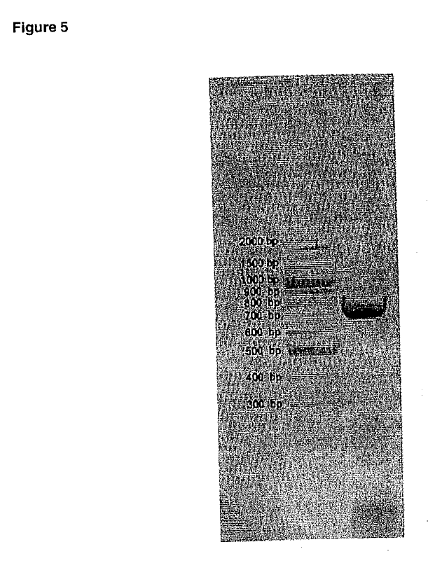

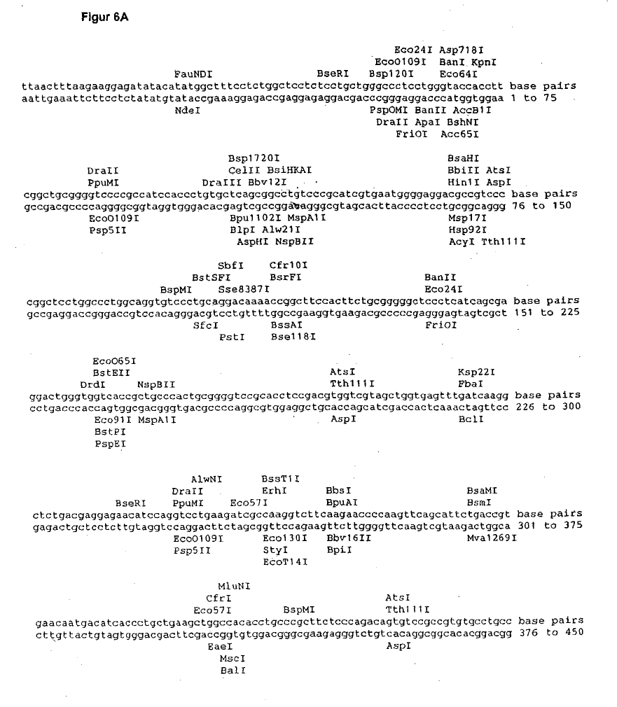

FIG. 5 shows the chymotrypsingen A-DNA manufactured in Example 2 following ligation by the Taq-ligase, exonuclease-treatment and PCR amplification. To the left is a 100 pb marker (New England Biolabs) coating, to the right 10 μl of the amplification preparation on a 1.5% agarose gel in 1× TBE.

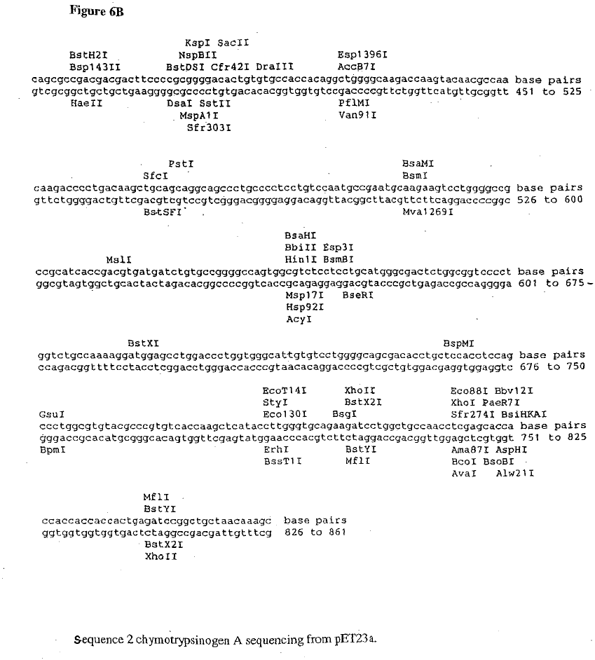

FIGS. 6A and 6B show the nucleotide sequence, identified through DNA-sequencing, of the DNA manufactured in Example 2 after cloning in the pET23a vector. The identified sequence for the gene for chymotrypsinogen A is identical to the desired sequence.

The present invention is illustrated in detail by the following examples.

### EXAMPLE 1

### Synthesis of the Xylanase Gene From A. kawachii

Manufacture

1. ODN-synthesis

All oligonucleotides (ODN) were synthesized according to the phosphoramidite method on an Expedite 8908 Synthesizer (formally Perseptive Biosystems). All chemicals used were provided by the firm of Proligo (Hamburg). The amidites used were absorbed in dry acetonitril (Proligo) (all components, including the phosphorylating reagent in a final concentration of 0.1 M) and dried prior to use via an activated molecular filter (Merck). To achieve optimally efficient synthesis of particularly long oligonucleotides, all coupling times were increased to 3 minutes. Dicyanoimidazol (Proligo) served as an activator for the coupling reaction. The CPG-support used had a pore diameter of 1000 Å (length<130 bp, Proligo) or O 2000 Å (length>130 pb, Glen Research). To obtain optimally complete 5′phosphorylated ODN's, the 5′end was reacted with the aid of [3-(4,4′-dimethoxytrityloxy)-2,2′-dicarboxyethyl]propyl-(2-cyanoethyl)-(N,N′-diisopropyl)-phosphoramidite (CPRII, Glen Research) following DMTr-on synthesis. In order to obtain correspondingly high coupling yields in this case as well, coupling times for this bonding were increased to 30 minutes. In this way, a total of 7 ODN's (Xyl1-Xyl7) were constructed for xylanase, among these 6 5-terminal phosphorylated (Xyl2-Xyl7), with an average length of 70-90 b (Table 1). For synthesis of the joint-DNA-oligonucleotides (gap fragments GXyl1-6), no further modifications were made to the synthesis protocols.

2. Purification

Once synthesized the base protection groups were then de-protected. This was done by transferring the support material (approximately 7 mg CPG) to a vessel with a threaded seal and treated for 24 h at 37° C. with a solution (500 μl) composed of three parts 32% ammoniac (Merck) and one part chilled ethanol (Fluka). Once the separation reaction is complete the preparation is then cooled over ice, and 100 μl of a 1M triethylammoniumacetate-solution (TEAA) are then added to the mixture. The entire sample is then separated by filtration from the support material and purified via RP-HPLC (column: 4.6 mm×300 mm packed with POROS R2 (Perseptive Biosystems); Buffer A: 100 mM TEAA, 5% acetonitril; Buffer B: acetonitril; flow: 4 ml/min; gradient: 40 columnar volumes of 0% to 50% Buffer B). The main fractions were trapped and dried in vacuum. Following detritylation with 80% acetic acid (30 minutes at 22° C.), the acetic acid was removed in vacuum and the remaining residue to be used for separating off the phosphate protection groups was treated for 15 minutes with 300 μl aqueous ammoniac solution (2 parts distilled water/conc. Ammoniac). Since there is no terminal modification (5′) to first base-DNA-oligonucleotide Xyl1, treatment of the bases was unnecessary. Next the de-protected oligonucleotides were precipitated with ethanol, absorbed in distilled water and analyzed via denaturizing PAGE (15%). The oligonucleotides were visualized using silver dye. There were no shortened sequences detectable for any of the oligonucleotides.

3. Gene Synthesis (Overview)

Gene synthesis may be subdivided into two steps. First is a ligation step in which the base-DNA-oligonucleotides (Xyl1-Xyl7, Table 1) are linked to one another with the aid of a ligase (e.g. Taq-ligase, T4-DNA-ligase or E. coli ligase) following hybridization to the short joint-DNA-oligonucleotides (gap fragments GXyl1-GXyl6). This partial step is generally referred to herein as oligonucleotide-specific ligation (OSL). Following OSL the entire reaction preparation is treated with exonuclease VII. In this process, all non-incorporated oligonucleotides, including the joint-DNA-oligonucleotides, are hydrolyzed. Next, a small portion of the hydrolase preparation is placed in a PCR with two primers (APXyl1 and APXyl7) binding terminally to the ODN's Xyl1 and Xyl7. This reaction produces the specific xylanase gene and simultaneously enables cloning in an appropriate plasmid by way of linker sequences introduced with APXyl1 and APXyl7.

4. Oligonucleotide-specific Ligation (OSL)

For the TSL, 2 μl each of the ODN's Xyl1-Xyl7 (10 μM) and 10 μl of the joint-DNA-oligonucleotides GXyl1-GXyl6 (10 μM) were mixed in a reaction vessel. The preparation was then mixed with 8.2 μl 10× ligase buffer (New England Biolabs) to which was added 2 μl (80U) Taq-DNA ligase (New England Biolabs). Subsequent incubation occurred at 37° C. for 12-14 h.

TABLE 1

Oligonucleotides for Constructing the Xylanase Gene

| | | Modi- |
|---|---|---|
| | | fica- |
| Name | Sequence (of 5′in 3′) | tion |
| Xy11 | t*a*g*g*c*aaattgggaattccatatgagtgctggtattaactacgtgcaaaactacaacggcaaccttgct | None |
| | gatttcacctatgacgagagtgtgccgga | |
| Xy12 | acattttccatgtactgggaagatggagtgagctccgactttgtcgttggtctgggctggaccactggttcttcgaatgctatcagctac | 5′ |

| | |
|---|---|
| tctg | phos- |

| | | |
|---|---|---|
| | | phate |
| Xy13 | ccgaatacagtgcttctggctcctcttcctacctcgctgtgtacggctgggttaactatcctcaggctgaatactacatcgtc | 5′ |
| | | phos- |
| | | phate |
| Xy14 | gaggattacggtgattacaacccttgcagctcggccacaagccttggtaccgtgtactctgatggaagcacctaccaagtctgcac | 5′ |
| | | phos- |
| | | phate |
| Xy15 | cgacactcgaactaacgaaccatcgatcacgggaacaagcacgttcacgcagtacttctccgttcgagagagcacgcgcacatctg | 5′ |
| | | phos- |
| | | phate |
| Xy16 | gaacggtgactgttgccaaccatttcaacttctgggcccagcatgggttcgggaattccgacttcaatta | 5′ |
| | | phos- |
| | | phate |
| Xy17 | tcaggtcatggcagtggaagcatggagcggcgccggcagcgccagtgtcacgatctcctctaaactcgagcggaat*t*a*a*t*t | 5′ |
| | | phos- |
| | | phate |
| GXy11 | gtacatggaaaatgttccggcactctcgtc | None |
| GXy12 | aagcactgtattcggcagagtagctgatag | None |
| GXy13 | atcaccgtaatcctcgacgatgtagtattc | None |
| GXy14 | ttagttcgagtgtcggtgcagacttggtag | None |
| GXy15 | caacagtcaccgttccagatgtgcgcgtgc | None |
| GXy16 | actgccatgacctgataattgaagtcgcta | None |
| APXy11 | aattgggaattccatatg | None |
| APXy17 | aattaattccgctcgagt | None |

*phosphorthioate bonds

5. Exonuclease Treatment

The entire ligation preparation was first precipitated with 50 μl 3M sodium acetate (pH 5.2) and 500 μl chilled ethanol on ice. Following precipitation the residue was dried in vacuum and dissolved in 50 μl distilled water. Added to the preparation was 50 μl exonuclease VII (20U, Pharmacia Biotech) in 100 mM Tris-HCL pH8.0, 400 mM NaCl and the entire preparation incubated 45 minutes at 37° C. The nuclease preparation was then extracted 1× using phenol-chloroform and 2× using chloroform and the aqueous residue transferred to a sterile cap.

6. PCR

For targeted amplification of the single stranded gene thus assembled, 2 μl of the nuclease preparation were mixed with 10 μl each of external primer APXyl1 (10 μM) and APXyl7 (10 μM), 8 μl dNTP-Mix (1.25 mM/dNTP), 5 μl 10× polymerase buffer (New England Biolabs) and 13 μl distilled water, and heated for 5 minutes at 95° C. Then the mixture was chilled on ice, mixed with 2 μl (4U) Vent Polymerase (New England Biolabs) and placed in the thermocycler (Hybaid) at 40° C. (addition). Subsequent amplification of the xylanase occurred under the following conditions:

TABLE 2
PCR-Conditions

| Step | Temperature | Time |
|---|---|---|
| Addition | 40° C. | 30 sec. |
| Extension | 72° C. | 1 min. |
| Denaturization | 95° C. | 30 sec. |
| Cycle count 35 | | |

Upon completion of PCR, the entire reaction preparation was mixed with 5× sample buffer and purified by means of gel electrophoresis (3% agarose) (FIG. 3). The xylanase gene was isolated following electroelution of the agarose bands cut from the gel (Sambrook et al., 1989, Molecular Cloning-A Laboratory Manual).

7. Cloning

After elution and extraction (Sambrook et al., 1989, Molecular Cloning-A Laboratory Manual), then immersion in TE buffer (10 mM tris-HCL, 0.5 mM EDTA pH8.0), the xylanase gene was completely digested with the restriction enzymes Ndel (New England Biolabs) and Xhol (New England Biolabs) over night at 37° C. and re-isolated using gel electrophoresis. Following electroelution and processing, the fragment was ligated over night at 16° C. in a suitably opened pET23a vector (Novagen). Ligation was performed using 200U T4 DNA ligase. Next, 5 μl of the ligation preparation was transformed in competent cells (DH5α) (Sambrook et al., 1989, Molecular Cloning-A Laboratory Manual).

8. Sequencing

Sequencing performed according to Saenger on an arbitrarily isolated clone produced a complete correspondence with the designed sequence (FIG. 4A and 4B).

### EXAMPLE 2

### Synthesis of Gene for Human Chymotrypsinogen A

1. ODN Synthesis

All oligonucleotides (ODN) were synthesized in accordance with the phosphoramidite method on an Expedite 8908. All chemicals and synthesis protocols used are identical to those indicated in paragraph 1 of Example 1. A total of 11 ODN's (Ch1-Ch11) were used to assemble the chymotrypsinogen-DNA, among these 10 5′-terminally phosphorylated (Ch2-Ch11), with an average length of 90 b (Table 3). In this example too, no further modifications were made to the synthesis protocols for synthesizing the short gap fragments (GCh1-10).

2. Purification

Once synthesized, the base protection groups were then de-protected. This was done by transferring the support material (approximately 7 mg CPG) to a vessel with a threaded seal and treated for 24 h at 37° C. with a solution (500 μl) composed of three parts 32% ammoniac (Merck) and one part chilled ethanol (Fluka). Once the separation reaction is complete the preparation is then cooled over ice, and 100 μl of a 1M triethyl ammonium acetate-solution (TEAA) are then added to the mixture. The entire sample is then separated by filtration from the support material and purified via RP-HPLC (column: 4.6 mm×300 mm packed with POROS R2 (Perseptive Biosystems); Buffer A: 100 mM TEAA, 5% acetonitril; Buffer B: acetonitril; flow: 4 ml/min; gradient: 40 columnar volumes of 0% to 50% Buffer B). The main fractions were trapped and dried in vacuum. Following detritylation with 80% acetic acid (30 minutes at 22° C.), the fractions were again rotated to dryness and, for purposes of separating off the phosphate protection groups, the residue was treated for 15 minutes with 300 μl aqueous ammoniac solution (2 parts distilled water/conc. ammoniac. Finally, the de-protected oligonucleotides were precipated with ethanol, immersed in distilled water and analyzed via denaturizing PAGE.

3. Gene synthesis

As in the case of the xylanase gene, the total synthesis of the gene for chymostrypsinogen A performed herein may be subdivided into two partial steps First, the long base-DNA-oligonucleotides (Ch1-11, Table 3) are linked to one another through OSL. After OSL the entire reaction preparation is treated with exonuclease VII. In the process, all non-incorporated oligonucleotides, including the joint-DNA-oligonucleotides, are hydrolyzed. A small portion of the hydrolase preparation is then placed in a PCR with two primers (APCh1 and APCh11) binding terminally to the ODN's Ch1 and Ch11. This reaction also specifically produces the chymotrypsinogen A-gene.

TABLE 3

Oligonucleotides for Constructing the Gene for Chymotrypsinogen A

| | | |
|---|---|---|
| Name | Sequence (of 5′ in 3′) | Modification |

| | | |
|---|---|---|
| Ch1 | a*t*g*g*a*tttcctcggctcctctcctgctgggccctcctgggtaccaccttcggctgcggggtccccgccatccaccct | None |
| CH2 | gtgctcagcggactgtcccgcatcgtgaatggggaggacgccgtccccggctcctggccctggccctggcaggtgtccctg | 5′phosphate |
| CH3 | caggacaaaaccggcttccactctgcgggggctccctcatcagcgaggactgggtggtcaccgctgcccactgcggg | 5′phosphate |
| CH4 | gtccgcacctccgacgtggtgctagctggtgagtttgatcaaggctctgacgaggagaacatccaggtcctg | 5′phosphate |
| CH5 | aagatcgccaaggtcttcaagaaccccaagttcagcattctgaccgtgaacaatgacatcaccctgctgaagctg | 5′phosphate |
| CH6 | gccacacctgcccgcttctcccagacagtgtccgccgtgtgcctgcccagcgccgacgacgacttccccgcg | 5′phosphate |
| CH7 | gggacactgtgtgccaccacaggctggggcaagaccaagtacaacgccaacaagacccctgacaagctgcag | 5′phosphate |
| CH8 | caggcagccatgcccctcctgtccaatgccgaatgcaagaagtcctggggccgccgcatcaccgacgtgatg | 5′phosphate |
| CH9 | atctgtgccggggccagtgccgtctcctcctgcatgggcgactctggcggtcccctggtctgccaaaag | 5′phosphate |
| CH10 | gatcgagcctggtccctggtgggcattgtgtcctggggcacgcacacctgctccacctttagccctggcgtg | 5′phosphate |
| CH11 | tacgcccgtgtcaccagctcataccttgggtgcagaagatcctggctg*c*c*a*a*c | 5′phosphate |
| GCh1 | caggccgctgagcacagggtggatggcggg | |
| GCh2 | gccggttttgtcctgcagggacacctgcca | |
| GCh3 | gtcggaggtgcggtaccccgcagtgggcag. | |
| GCh4 | gaccttggcgatcttcaggacctggatgtt | |
| GCh5 | gcgggcaggtgtggccagcttcagcagggt | |
| GCh6 | ggcacacagtgtccccgcggggaagtcgtc | |
| GCh7 | gggcagggctgcctgctgcagcttgtcagg | |
| GCh8 | ggccccggcacagatcatcacgtcggtgat | |
| GCh9 | ggtccaggctccatcctttggcagaccag | |
| GCh10 | ggtgacacgggcgtacacgccagggctgga | |
| APCh1 | gggaattccatatggctttcctctgg | |
| APCh1 | ccgctcgagttggcagccaggatcttc | |

*phosphorthioate bonds

4. Oligonucleotide-Specific Ligation (OSL)

For the TSL 8 μl each of the ODN's Ch1-Ch11 (10 μM) and 4 μl of the joint-DNA-oligonucleotides GCh1-GCh10 (10 μM) were mixed in a reaction vessel. It is also feasible to use a mixing ratio of 1:1 to 1:10. The preparation was then mixed with 8.2 μl 10× ligase buffer (New England Biolabs) to which was added 2 μl (8U) Taq-DNA ligase (New England Biolabs) and filled to 80 μl with distilled water. Subsequent incubation occurred at 37° C. for 12-14 h.

5. Exonuclease Treatment

As in Example 1 the entire ligation sample was first precipitated with 50 μl 3M sodium acetate (pH 5.2) and 500 μl chilled ethanol on ice. After precipitation the residue was dried in vacuum and dissolved in 50 μl distilled water. To the preparation was then added 50 μl exonuclease VII (20U, Pharmacia Biotech) in 100 mM Tris-HCL pH8.0, 400 mM NaCl and the entire preparation incubated 45 minutes at 37° C. The nuclease preparation was then extracted using phenol-chloroform and the aqueous residue was transferred to a sterile cap.

6. PCR

To amplify the gene assembled by OSL, 2 μl of the nuclease preparation were combined with 10 μl each of external primer APCh1 (10 μM) and APC11 (10 μM), 8 μl dNTP-mix (1.25 mM/dNTP), 5 μl 10× polymerase buffer (New England Biolabs) and 13 μl distilled water, mixed, then heated for 5 minutes at 95° C. The mixture was then chilled on ice, mixed with 2 μl (4U) Vent Polymerase (New England Biolabs) and placed in the thermocycler (Hybaid) at 54° C. (addition). Subsequent amplification of the xylanase occurred under the following conditions:

TABLE 4
PCR-Conditions

| Step | Temperature | Time |
|---|---|---|
| Addition | 54° C. | 30 sec. |
| Extension | 72° C. | 1 min. |
| Denaturization | 94° C. | 30 sec. |
| Cycle count 35 | | |

Upon completion of PCR, the entire reaction preparation was mixed with 5× sample buffer and purified by means of gel electrophoresis (3% agarose) (FIG. 5). The chymotrypsinogen-DNA was isolated following electroelution of the agarose bands cut from the gel (Sambrook et al., 1989, Molecular Cloning-A Laboratory Manual).

7. Cloning

Following elution and extraction (Sambrook et al., 1989, Molecular Cloning-A Laboratory Manual), then immersion in TE buffer (10 mM tris-HCL, 0.5 mM EDTA pH8.0), the chymotrypsinogen-DNA was completely digested with the restriction enzymes Ndel (New England Biolabs) and Xhol (New England Biolabs) over night at 37° C. and re-isolated by gel electrophoresis. Following electroelution and processing, the fragment was ligated over night at 16° C. in a suitably opened pET23a vector (Novagen). Ligation was performed using 200U T4 DNA ligase (New England Biolabs). Next, 10 μl of the ligation preparation were transformed in competent cells (DH5α) (Sambrook et al., 1989, Molecular Cloning-A Laboratory Manual).

8. Sequencing

Sequencing performed according to Saenger on an arbitrarily isolated clone produced a complete correspondence with the designed sequence (FIG. 6A and 6B).

Cited References

Au, L. C. et al. (1998) Biochem. Biophys. Res. Commun., 248,200-203

Ausubel, F. M. et al. (1994) Current protocols in molecular biology, Vol 1. Wiley

Casimiro, D. R. et al. (1997) Structure, 5,1407-1412

Chalmers, F. M. & Curnow, K.M. BioTechniques, 30,249-252

Chang, H. H. (1993) Biochem. Biophys. Res. Commun., 190,242-249

Chen, H. B. (1990) Nucleic Acid Res., 18, 871-878

Ciccarelli, R. B. et al. (1991) Nucl. Acids. Res., 19,6007-6013

Climie, S. et al. (1990) Proc. Natl. Acad. Sci. USA, 87,633-637

Dillon, P. J. & Rosen, C. A.(1990) BioTechniques, 9,298-300

Ferretti, L. (1986) Proc. Natl. Acac. Sci. USA, 87,599-603

Hostomsky, Z et al. (1987) Nucl. Acids Res., 15,4849-4856

Jayaraman, K. et al. (1991) Proc. Natl. Acad. Sci.USA, 88,4084-4088

Jayaraman, K. & Puccini, C. J. (1992) BioTechniques, 12,392-398

Oprian, D. D. et al. (1991)

Biochemistry, 30, 11367-11372

Spoat, B. S. et al. (1985) Nucl. Acids Res., 13,2959-2977

Traub, P. C. et al. (2001) Appl. Microbiol. Biotechnol. (2001), 55,198-204

Uhlmann, E & Hein, F. (1987) NucleicAcids Symp Ser. 18,237-240

Wosnick, M. A. (1989) Gene, 76,153-160

Ye, Q. Z. (1992) Biochem, Biophys. Res. Commun., 186,143-149

1. A method for manufacturing DNA comprising the steps of
a) preparing n single stranded base-DNA-oligonucleotides which form immediately consecutive parts of the nucleotide sequence of the DNA being manufactured, in which
i) the second to the n-th base-DNA-oligonucleotide is phosphorylated at the 5′end and
ii) n is at least 2;
b) preparing at least (n−1) single stranded joint-DNA-oligonucleotides, applicable to which joint-DNA-oligonucleotide, the 3′terminal region of a joint-DNA-oligonucleotide is at least partially complementary to the 3′-terminal region of a base-DNA-oligonucleotide, and the 5′-terminal region of the same joint-DNA-oligonucleotide is at least partially complementary to the 5′-terminal region of the immediately following base-DNA-oligonucleotide, so that when a joint-DNA-oligonucleotide is hybridized with 2 immediately consecutive base-DNA-oligonucleotides a double-stranded DNA-hybrid is formed in the region of the joint-DNA-oligonucleotide;
c) contacting the base-DNA-oligonucleotides with the joint-DNA-oligonucleotides;
d) subjecting the product DNA-hybrid from step c) to a ligation reaction;
e) subjecting the reaction product from step d) to an exonuclease reaction, in which the DNA strand of the reaction product of step d) formed by ligated base-DNA-oligonucleotides includes at least two cap-structures.

2. The method according to claim 1, characterized in that the reaction product from step e) is further subjected to a PCR.

3. The method according to claim 2, characterized in that in said PCR a first primer is used that has a target sequence located in the region of the first base-DNA-oligonucleotide, and a second primer is used that has a target sequence located in the region of the n-th base-DNA-oligonucleotide.

4. The method according to claim 2 or 3, characterized in that in said PCR primers are used which contain one or more recognition sequences for one or more restriction endonucleases.

5. The method according to claim 2 or 3, characterized in that the double stranded reaction product of the PCR is further subjected to restriction digestion.

6. The method according to claim 4, characterized in that the double stranded reaction product of the PCR is further subjected to restriction digestion.

7. The method according to any one of claims 2, 3, or 6, characterized in that the ligation reaction is carried out using a ligase selected from the group consising of T4-DNA-ligase, Taq DNA-ligase and Pfu-ligase.

8. The method according to claim 4, characterized in that the ligation reaction is carried out using a ligase selected from the group consising of T4-DNA-ligase, Taq DNA-ligase and Pfu-ligase.

9. The method according to claim 5, characterized in that the ligation reaction is carried out using a ligase selected from the group consising of T4-DNA-ligase, Taq DNA-ligase and Pfu-ligase.

10. The method of claim 1, characterized in that the exonuclease reaction is carried out using an enzyme selected from the group consisting of exonuclease VII, general exonuclease, preferably exonuclease VII, but also exonuclease I, exonuclease III and exonuclease V, as well as DNase 1 and mixtures of the aforementioned hydrolases.

11. The method of claim 1, characterized in that the cap-structure is selected from the group consisting of thioate bonds between individual nucleotides, 2′Omethyl-RNA, modified bases, DNA-sequences with loop structure(s) and RNA-sequences with loop structure(s).

12. The method of claim 1, characterized in that said first base-DNA-oligonucleotide includes a cap-structure and said n-th base-DNA-oligonucleotide includes a cap-structure.

13. The method of claim 1, characterized in that said base-DNA-oligonucleotides and/or the joint-DNA-oligonucleotides are produced by way of the phosphoramidite method.

14. The method of claim 1, characterized in that one or more base-DNA-oligonucleotides and/or joint-DNA-oligonucleotides contain randomized nucleotides.

15. The method of claim 1, characterized in that the manufactured DNA is further cloned in a vector or a plasmid.

16. The method of claim 1, characterized in that the manufactured DNA or the manufactured vector or the manufactured plasmid is introduced into a cell.

17. A DNA obtained by the method of claim 1.

18. A DNA manufactured in accordance with the method of claim 16.

19. A DNA hybrid comprising a single strand, one or more joint-DNA-oligonucleotides hybridized therewith, a cap-structure in the 5′-terminal region of the single strand and a cap-structure in the 3′-terminal region of the single strand.

20. A kit for manufacturing DNA which contains a first base-DNA-oligonucleotide that includes a cap-structure, a second base-DNA-oligonucleotide that includes a cap-structure, an enzyme exhibiting ligase activity and an enzyme exhibiting exonuclease activity.

21. A kit according to claim 20, characterized in that said kit also contains means for performing a PCR.

22. Kit according to claim 21, characterized in that said kit contains a thermostable DNA-polymerase and primers which contains one or more recognition sequences for one or more restriction endonucleases.
